(19) United States
(12) Patent Application Publication
(10) Pub. No.: US 2024/0082433 A1
(43) Pub. Date: Mar. 14, 2024

(54) TUMOR RESECTION PROCESS AND COMPOSITION CONTAINING IRIDIUM-CONTAINING NUCLEOSIDE

(71) Applicant: Cleveland State University, Cleveland, OH (US)

(72) Inventor: Anthony J. Berdis, Chesterland, OH (US)

(73) Assignee: Cleveland State University, Cleveland, OH (US)

(21) Appl. No.: 18/235,920

(22) Filed: Aug. 21, 2023

Related U.S. Application Data
(60) Provisional application No. 63/399,293, filed on Aug. 19, 2022.

(51) Int. Cl. A61K 49/00; A61B 5/00

(52) U.S. Cl. CPC A61K 49/0015; A61B 5/0071; A61B 5/4887; A61B 5/4064; A61B 5/4312; A61B 5/4325

(57) ABSTRACT

A tumor resection process includes the administration of an iridium-containing nucleoside, medical imaging, and tumor and tumor margin removal. The iridium-containing nucleoside may be selected from:

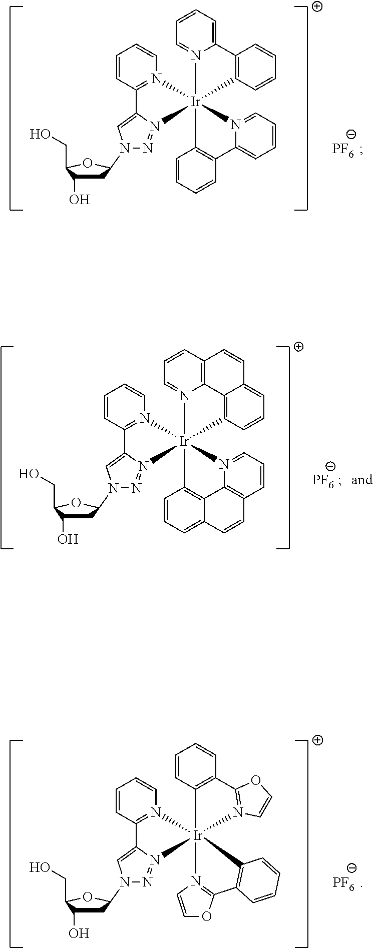

This application claims the priority benefit of U.S. Provisional Application Ser. No. 63/399,293, filed Aug. 19, 2022, the contents of which are incorporated by reference herein.

### BACKGROUND

20 million people in the United States are currently living with cancer. The first treatment option for most cancer patients involves surgical excision of solid tumors to reduce tumor mass. Surgical excision is used in approximately 50% of all cancer patients. The clear goal of surgery is to remove all cancerous material in addition to removing a border of healthy tissue known as the tumor margin. Achieving a cancer-free tumor margin is important for defining successful surgical interventions, especially in cancers such as brain, lung, and breast in which surgical removal of healthy, normal tissues must be minimized to avoid devastating effects on cancer patients. Unfortunately, a large percentage of cancer surgeries ultimately fail due to the inability to define an accurate tumor margin during the surgical procedure. This deficiency occurs since most imaging agents used in surgical oncology suffer from low sensitivity and low selectivity for detecting cancer cells in real time. These deficiencies reduce the ability of surgeons to accurately define tumor margins, and this represents a significant health risk for cancer patients.

Spectroscopic techniques such as X-ray, MRI, and CT scanning have improved the ability of surgical oncologists to locate and estimate the size of a tumor prior to surgery. Unfortunately, pre-operative imaging does not always produce successful surgical resections since tissue often shifts prior to and during surgery. In addition, small clusters of cancer cells frequently dissociate from the primary mass. Since these cancerous cells are not easily visible, they remain in the patient to cause tumor recurrence. Although MRI can be used during surgery to provide “real-time” images of dissociated cancer cells, this procedure is not routinely used as it is cumbersome and time-consuming.

A more practical approach is to employ fluorescent dyes that can distinguish cancerous from normal tissue. After exposure to particular wavelengths of light, cameras capture the fluorescence emitted by the dye within cancer cells and depict it on monitors to provide surgeons with “real-time” images of the tumor margin. Several compounds are currently for this purpose. For example, 5-aminolevulinic acid (5-ALA) was the first agent used in the surgical resection of malignant gliomas. 5-ALA is a precursor in the hemoglobin biosynthesis pathway and when delivered in high concentrations results in the accumulation of protoporphyrin IX (PpIX). PpIX absorbs blue light (˜400 nm) and emits in the red range (˜640 nm). Cells that emit red fluorescence are defined as cancerous. Unfortunately, 5-ALA is not highly selectivity for cancerous versus normal tissues, and this diminishes the efficacy of this imaging agent to accurately define tumor margins. Collectively, low sensitivity coupled with low selectivity of current imaging agents remains a significant problem to accurately define tumor margins.

### BRIEF DESCRIPTION

The present disclosure relates to iridium-containing nucleosides and the administration of the same for diagnostic and therapeutic purposes. Cancer treatment processes are also disclosed.

Disclosed, in some embodiments, is a process of treating a cancer patient. The process includes: administering an iridium-containing nucleoside to the patient; obtaining a medical image of the patient; and excising a tumor and a tumor margin from the patient, wherein an amount of tissue excised is based on identifying cellular uptake of the iridium-containing nucleoside from the medical image.

The medical image may be obtained before or simultaneously with the excising element. In particular embodiments, medical imaging is provided in real time during surgery.

In some embodiments, the iridium-containing nucleoside is selected from:

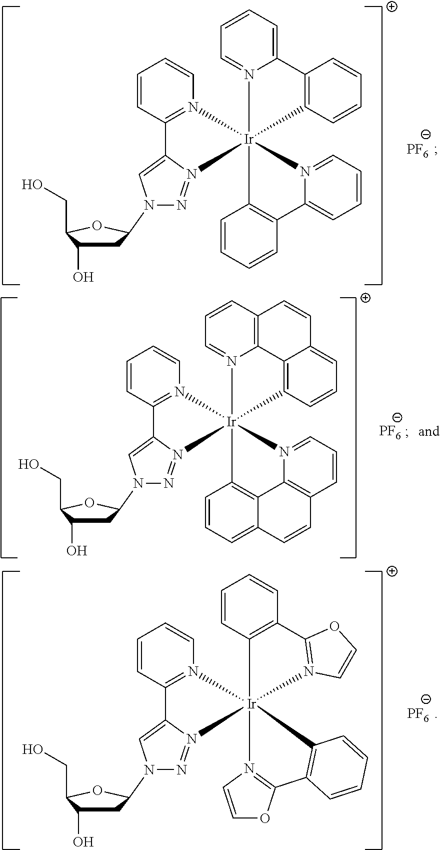

There may be a waiting period of between about 4 and about 24 hours after administration of the iridium-containing nucleoside to allow for cellular uptake.

In some embodiments, the iridium-containing nucleoside is a structural analog of a naturally occurring deoxynucleoside (e.g., adenosine).

The iridium-containing nucleoside may include a pyridyl triazole linking iridium to deoxyribose.

In some embodiments, the tumor is a brain tumor, a breast tumor, or an ovarian tumor.

Disclosed, in other embodiments, is an iridium-containing nucleoside selected from:

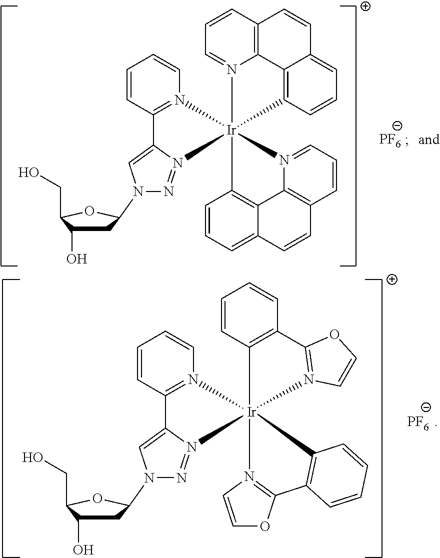

These and other non-limiting characteristics are more particularly described below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following is a brief description of the drawings, which are presented for the purposes of illustrating the exemplary embodiments disclosed herein and not for the purposes of limiting the same.

FIG. 1 is a flow chart illustrating a non-limiting example of a process in accordance with some embodiments of the present disclosure.

FIG. 2 illustrates the chemical structures of Ir(III)-PPY nucleoside (left) and the natural nucleoside, deoxyadenosine (right).

FIG. 3 includes fluorescence microscopy images of the dose-dependent uptake of Ir(III)-PPY nucleoside in the human cancer cell line, KB3-1, particularly showing increased Ir(III)-PPY nucleoside (green) as a function of increasing concentration (Δt=4 hours).

FIG. 4 includes microscopy images showing co-localization of Ir(III)-PPY nucleoside in glioblastoma cells expressing red-fluorescent protein (RFP-U87).

FIG. 5 illustrates the chemical structures of Ir(III)-PPY nucleoside (left); Ir(III)-BZQ nucleoside (center); and Ir(III)-PBO nucleoside (right).

FIG. 6 includes fluorescent microscopy images of 3D brain cancer masses treated with various iridium-containing nucleosides (1 μM) for 24 hours.

FIG. 7 illustrates the time-dependent uptake and cell-killing effects of Ir(III)-BZQ nucleoside (10 μM) against 2D and 3D brain cancer cells.

FIG. 8 includes close-up merged microscopy images (transmitted/green/red) of 2D and 3D brain cancer cells treated with 10 μM Ir(III)-BZQ nucleoside for 1 vs. 3 days.

FIG. 9 includes microscopy images as discussed in the Examples.

FIG. 10 includes microscopy images as discussed in the Examples.

FIG. 11 includes microscopy images as discussed in the Examples.

FIG. 12 includes microscopy images as discussed in the Examples.

FIG. 13 includes gel electrophoresis images as discussed in the Examples.

FIG. 14 includes microscopy images as discussed in the Examples.

### DETAILED DESCRIPTION

The present disclosure may be understood more readily by reference to the following detailed description of desired embodiments included therein. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All publications, patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps. However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant figures and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The present disclosure relates to a new technological platform that can differentiate cancer cells from normal cells using metabolic differences associated between cancerous and non-cancerous cells. The key element relies on the use of Iridium-containing nucleoside analogs that are efficiently and selectively imported into several types of cancer cells compared to their normal counterparts. The unique fluorogenic properties of this class of analog provides an accurate, sensitive, and convenient method to directly identify and quantify the size of three-dimensional structures of cancerous masses need for proper surgical resection.

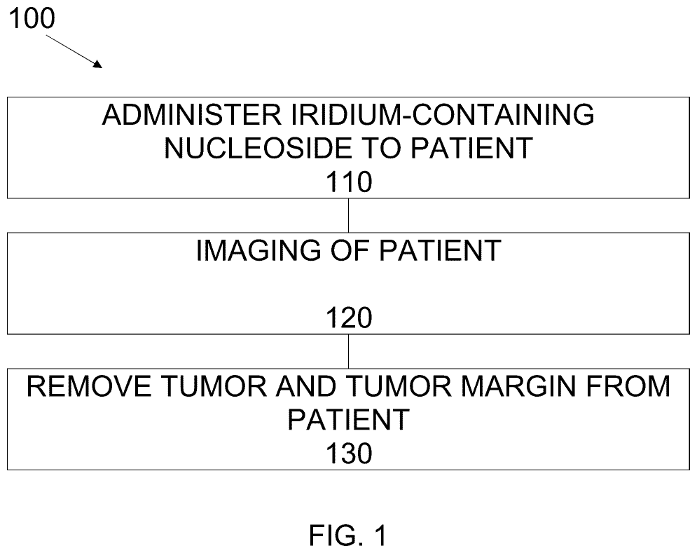

FIG. 1 is a flow chart illustrating a non-limiting example of a cancer treatment process 100 in accordance with some embodiments of the present disclosure. The process 100 includes administering an iridium-containing nucleoside to a patient 110, imaging the patient 120, and removing a tumor and tumor margin from the patient 130.

Administration 110 may be orally, intravenously, intramuscularly, or locally at the tumor site.

The iridium-containing nucleoside may be a structural analog of deoxyadenosine. In some embodiments, the iridium-containing nucleoside includes a pyridyl triazole linking iridium to deoxyribose.

Non-cancerous and cancerous cells may uptake the iridium-containing nucleoside at different rates (i.e., the rate of cellular uptake into cancer cells may be significantly higher).

Medical imaging 120 is performed around the tumor site. Fluorescence guided resection may be performed using imaging devices that provide real time information simultaneously by combining color reflectance images (bright field) with fluorescence emission from the compound. One or more light sources are used to excite and illuminate the compound in tumors. Light is collected using optical filters that match the emission spectrum of the fluorogenic compound. Imaging lenses and digital cameras such as charged-coupled device (CCD) and complementary metal-oxide semiconductor (CMOS) are used to produce the final image. Live video processing is employed to enhance contrast during fluorescence detection to improve signal-to-background ratios.

Fluorescence excitation can be achieved using a variety of light sources. Standard practices employ halogen lamps to deliver high power intensity with different band pass filters to generate different excitation channels ranging from ultraviolet to near infrared. Light-emitting diodes (LEDs) can also be used for broad band illumination and narrow band excitation. Both halogen lamps and LEDs are suitable for white light illumination during surgery. Excitation can also be performed using laser diodes when high power over a short wavelength range (typically 5-10 nm) is needed.

Live images using fluorescent dye and the surgical field are obtained using a combination of filters, lenses and cameras. During open surgery, hand-held devices are typically used due to their ease of use and mobility. In some cases, a stand can be used to maintain the system on top of the operating field, particularly when the weight and complexity of the device is high, i.e. use of multiple cameras.

Fluorescence guided resection can also be performed using minimally invasive devices such as endoscopes and laparoscopes. These approaches use a system of filters, lenses and cameras attached to the end of the probe. Unlike open surgery, the background from external light sources is reduced. However, the excitation power density at the sample is limited by the low light transmission of the fiber optics particularly in the near infrared.

An imaging system similar to Quest Spectrum that has a dynamic range for excitation/emission wavelengths may be utilized. The instrument can also provide real-time overlay images of fluorescence/transmitted data. The working distance and field of view allow surgeons to detect tumors ranging in size from 10 mm³ to 1 cm³ (microscopic detection) and 1 cm³ to 10,000 cm³.

Pre-operative and operative imaging are possible. However, this option will depend on the type of cancer. For example, the nucleoside could be used for pre-operative diagnostic imaging and tumor margin detection in cancers that can be viewed by endoscopy. These include esophageal, lung, and colon cancer. In other types of tumors such as neurological cancers, breast, ovarian, pancreatic, and liver, the nucleoside would probably be used only during operative procedures to define tumor margins. The nucleoside could be used for follow-up imaging in such cases.

In some embodiments, the imaging includes pre-operative, operative, and post-operative imaging. In other embodiments, the imaging includes any combination of two of these three imaging techniques. In further embodiments, the imaging includes only of these three types of imaging techniques.

Generally, a waiting period occurs between the administration 110 and the imaging 120 to allow for cellular uptake of the iridium-containing nucleoside into cancer cells prior to the imaging. The waiting period may be in a range of about 4 to about 24 hours, from about 2 to about 8 hours, or from about 1 to about 4 hours. The iridium-containing nucleoside fluoresces, thereby allow cancer cells to be distinguished from healthy cells.

The tumor and tumor margin are excised 130 from the patient based on the fluorescence. Excision 130 may be performed after imaging 120. However, in preferred embodiments, real-time imaging 120 is performed during surgery to excise 130 the tumor and tumor margin. Real-time imaging may be helpful where there is concern of cancer cells separating and/or tumor movement during the surgery.

The fluorescence of the iridium-containing nucleoside allows the surgeon to accurately locate the edges of the tumor. In addition to the tumor, a tumor margin is removed to ensure removal of all cancerous tissue. In some embodiments of the present disclosure, typical tumor margins are between 1 to 2 mm. In some embodiments, the tumor margin is less than 1 mm. Tumor margins are typically measured shortly after they are removed by surgery using histopathology to differentiate cancerous from normal tissue. It usually takes long periods of time (>1 hour) to accomplish this. However, the systems and methods of the present disclosure may reduce this time.

The iridium-containing nucleoside may also be administered for purely diagnostic purposes. Diagnostic methods include administering the iridium-containing nucleoside and subsequently conducting medical imaging after a waiting period to allow for cancer cell uptake of the iridium-containing nucleoside.

Additionally, the iridium-containing nucleoside may exhibit cancer cell killing effects.

The Iridium nucleoside can detect cancerous masses (3D masses) at concentrations in the high nanomolar range.

3D masses of cancer cells can be detected and their size defined within 24 hours of treatment.

The Iridium-containing nucleoside can detect cancerous cells (2D) at concentrations in the low micromolar range (1-10 μM). The concentration range depends on several factors. The first is the time frame the cells are treated with the analog (short times require higher concentrations whereas longer incubation times require lower concentrations). Another factor is the type of cancer being tested. Some cancers have high expression levels of a nucleoside transporter (hENT1) which is responsible for uptake of the nucleoside analog. Thus, these cancers show more efficient uptake compared to cells with lower expression levels of hENT1.

The Iridium nucleoside can distinguish between cancerous and normal cells.

The Iridium nucleoside show time-dependent cell-killing effects against a variety of cancer cells (brain, breast, and ovarian).

The Iridium nucleoside can detect cancer masses in addition to eliminating residual cancer cells.

Criteria for selecting an effective nucleoside may include:
1. Efficient uptake in cancer cells (rapid uptake (<2 hours) at low nM concentrations).
2. Selective uptake in cancer cells/poor to no uptake in normal cells.
3. High fluorescence (red or near infrared) that is diffuse in cancer cells.

Analogs possessing aromatic functional groups (Ir(III)-PPY and Ir(III)-BZQ) may fit these criteria. Analogs that contain oxygen, nitrogen, and/or sulfur atoms in non-liganded positions may show poor uptake and/or accumulation in distinct organelles.

Furthermore, in some embodiments, the nucleoside is selected from the group consisting of:

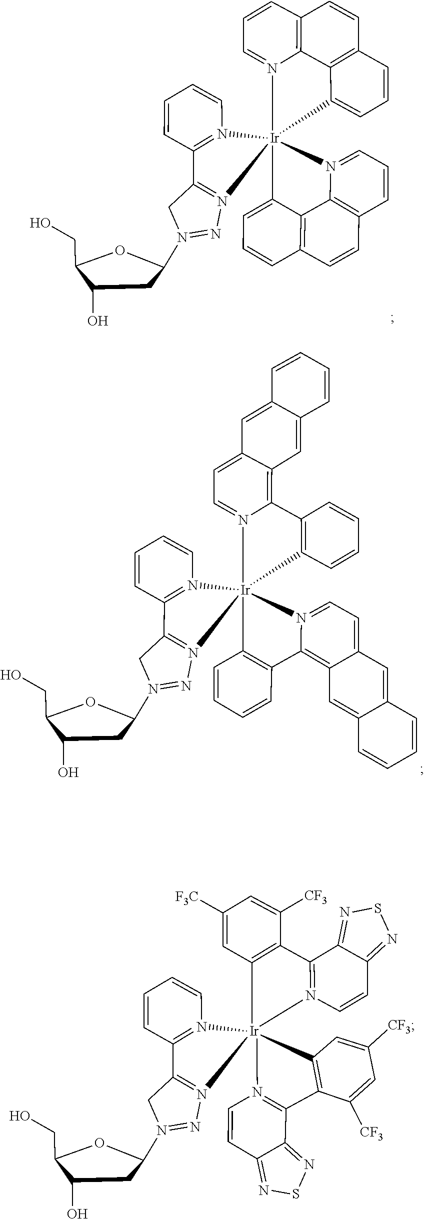

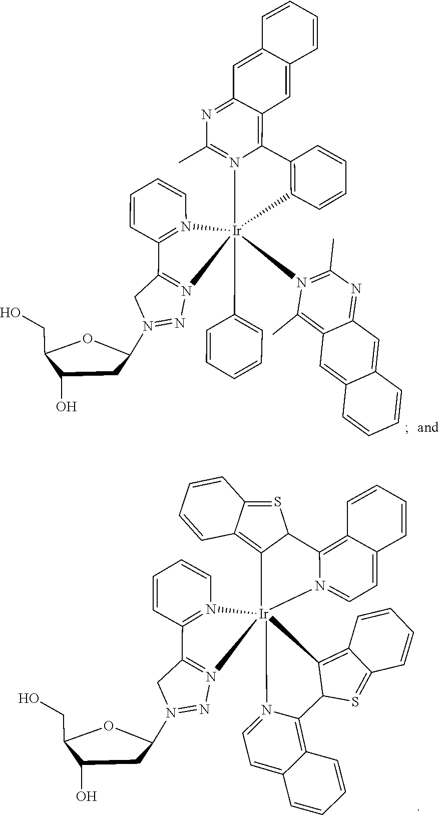

Although not shown, it should be understood that the above-depicted nucleosides are positively charged and used with a counterion. Non-limiting examples of counterions include [PF₆]⁻ and halide ions (e.g., Cl⁻).

Moreover, although various other formulas are depicted herein with the [PF₆]⁻ anion, it should be understood that this ion may be replaced with a different counterion (e.g., Cl⁻).

The combined diagnostic/therapeutic activities provide a practical solution for optimal surgical resection to prevent tumor recurrence.

The following examples are provided to illustrate the devices and methods of the present disclosure. The examples are merely illustrative and are not intended to limit the disclosure to the materials, conditions, or process parameters set forth therein.

### EXAMPLES

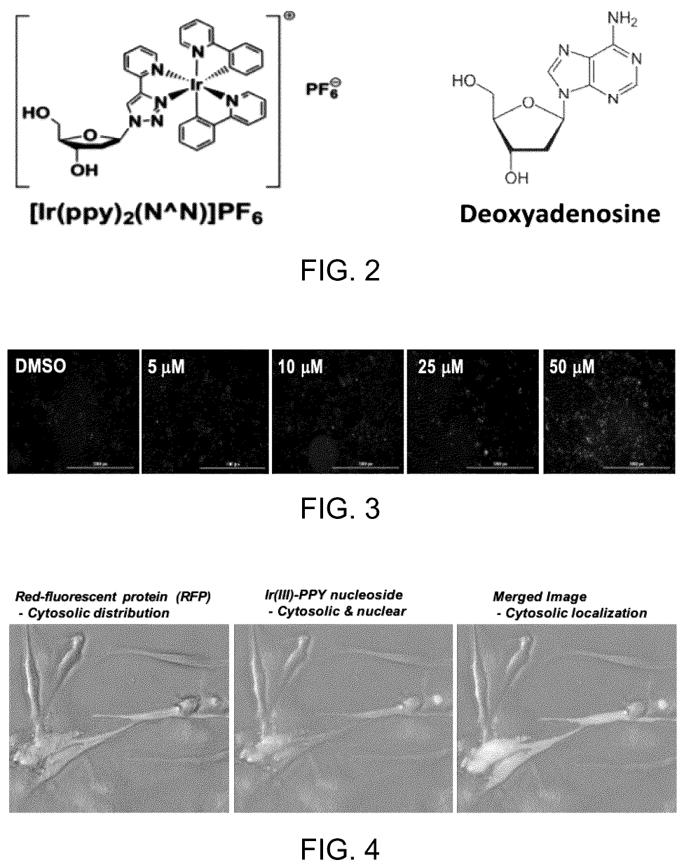

Several Iridium-containing nucleosides were synthesized and their imaging and anti-cancer activities were characterized. One non-limiting example of a compound is designated as Ir(III)-PPY nucleoside and shown in FIG. 2 next to deoxyadenosine. The synthetic design relies on the catalyzed [3+2] cycloaddition of azides to terminal alkynes. In this design, pyridyl triazoles covalently link the metal atom to the deoxyribose and this prevents metal dissociation from the complex. The cellular uptake of Ir(III)-PPY nucleoside into cervical cancer cells (KB3-1 cell line) was evaluated by treating cells with variable concentrations of Ir(III)-PPY nucleoside (0-50 μM). At times ranging from 4 to 24 hours, fluorescence microscopy images were taken using an excitation wavelength (λₑₓ) of 340 nm and an emission wavelength (λₑₘ) of 480 nm. FIG. 3 shows that the intracellular accumulation of Ir(III)-PPY nucleoside (green fluorescence) is dose-dependent. The uptake of low concentrations (10 μM) of Ir(III)-PPY nucleoside can be visualized as early as 4 hours post treatment. Ir(III)-PPY nucleoside produces weak cytotoxic effects (IC₅₀˜50 μM) after 24 hours. Thus, the cellular uptake can be measured on short time scales (Δt<12 hours) without complications arising from effects on cell viability.

Fluorescent microscopy was then used to validate that the uptake of Ir(III)-PPY nucleoside is selective for cancer cells. FIG. 4 compares the uptake of Ir(III)-PPY nucleoside using a human brain cancer cell line, U87, that expresses red fluorescent protein (RFP) versus normal fibroblasts. In these experiments, RFP-U87 cells were co-cultured with normal fibroblasts overnight and then treated with 10 μM Ir(III)-PPY nucleoside for 4 hours. As illustrated, Ir(III)-PPY nucleoside rapidly accumulates in U87 cancer cells whereas no uptake of the Iridium-containing nucleoside is observed in normal fibroblasts. Similar results were obtained at longer time intervals encompassing 24 hours incubation with Ir(III)-PPY nucleoside (data not shown). Collectively, the selective uptake of Ir(III)-PPY nucleoside in cancer cells suggests that the metal-containing nucleoside can better define tumor margins by accurately distinguishing cancerous cells from non-cancerous cells.

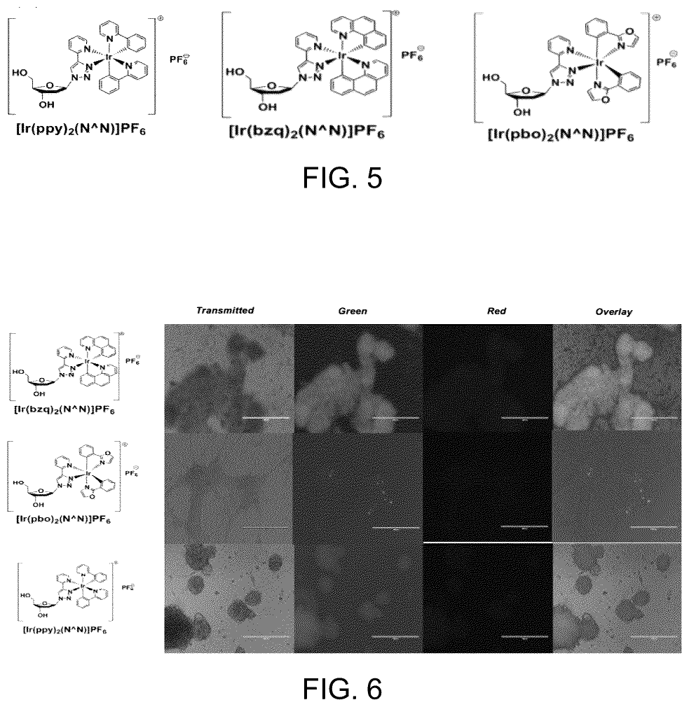

To improve the imaging capabilities of this class of compound, additional analogs corresponding to Ir(III)-BZQ nucleoside and Ir(III)-PBO nucleoside (FIG. 5) were synthesized and tested for uptake using 3-dimensional cell culture techniques. In these experiments, brain cancer cells (U87) were allowed to grow into defined 3D masses which can be distinguished from cells exhibiting morphological features of 2-dimensional cells. Fluorescent microscopy was used to investigate the uptake of each nucleoside in which cells were treated with 1 μM of each compound for 24 hours. Results displayed in FIG. 6 show that Ir(III)-BZQ nucleoside displays superior activity amongst the three compounds tested. This conclusion is based on the more intense fluorescent signal observed in cells treated with Ir(III)-BZQ nucleoside that accurately defines the overall 3-dimensional shape of the cancerous mass. In particular, the overall intensity in green fluorescence caused by the uptake of Ir(III)-BZQ nucleoside is about 5 times greater than observed in cells treated with equivalent concentrations of Ir(III)-PPY nucleoside. Equally important microscopy images from cells treated with Ir(III)-BZQ nucleoside display red fluorescence, and this is not observed in cells treated with either Ir(III)-PPY nucleoside or Ir(III)-PBO nucleoside. Finally, cells treated with Ir(III)-PBO nucleoside display unique punctates that correspond to uptake of the metal nucleoside into defined organelles within the cell. While this represents an interesting phenomenon, this behavior precludes the ability of Ir(III)-PBO nucleoside to function as a “tumor margin” imaging agents since there is no defined outline of the cancerous mass.

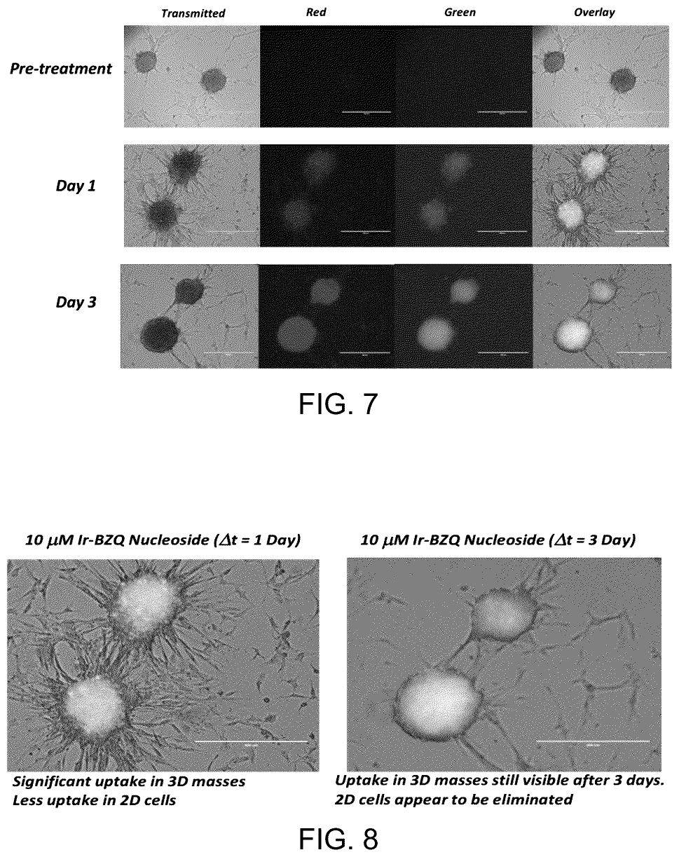

Based on these results, experiments focused more closely to define the diagnostic and therapeutic activities of Ir(III)-BZQ nucleoside. In these experiments, brain cancer cells (U87) were grown under conditions conducive with the formation of defined 3D masses in addition to cells exhibiting growth in 2 dimensions. Fluorescent microscopy techniques were used to measure both red and green fluorescence associated with the cellular uptake of the analog over a three (3) day treatment period. Images provided in FIG. 7 show that treatment with 10 μM Ir(III)-BZQ nucleoside generates intense red and green fluorescence signals after 1 day of treatment. The fluorescent signals are only observed in the 3D cancerous masses, thus making them easily identifiable at low magnification (10×). Little to no fluorescence signal is observed in 2D cells, and this likely reflects low magnification used in these experiments. Remarkably, after three days of treatment, the 3D masses still show intense fluorescence signals although the morphological features of these masses is different in comparison to treatment for 1 day. The change in morphological shape is most caused by the death of cells that reside at the outer fringe of the cancerous mass. This is more evident upon closer inspection of FIG. 8 which shows the changes in 3D masses in addition to the reduction in the number of 2D cells.

Collectively, these examples provide evidence for the ability of Iridium-nucleosides to accurately detect 3D cancerous masses that resemble tumors in human patients. The spectroscopic properties of these analogs will allow surgeons to identify tumor masses and distinguish them from normal surrounding tissue to better define the tumor margin. In addition, these novel analogs possess cell killing effects at low micromolar concentrations. This property will be valuable in eradicating any cancerous cells that remain at the surgical site to dramatically reduce the risk of tumor recurrence.

### Comparing the Theranostic Activity of Ir(III)-BZQ Nucleoside With Indocyanine Green (Gold Standard in Tumor Margin Detection Agents)

Indocyanine green is a strongly fluorescent dye with emission in the near-infrared (NIR) spectral range (700-900 nm). These properties, deep signal penetration and minimal interference of tissue autofluorescence, make Indocyanine green the gold-standard in NIR fluorescence image-guided surgery in oncology. More than 150 clinical trials have been conducted using Indocyanine green in several types of cancer including breast, colon, prostate, skin, and lung cancers. The goal here is to compare the fluorogenic and cell-killing properties of Ir(III)-BZQ nucleoside with Indocyanine green to determine if Ir(III)-BZQ nucleoside has similar or superior properties that would have improved surgical resection in cancer patients.

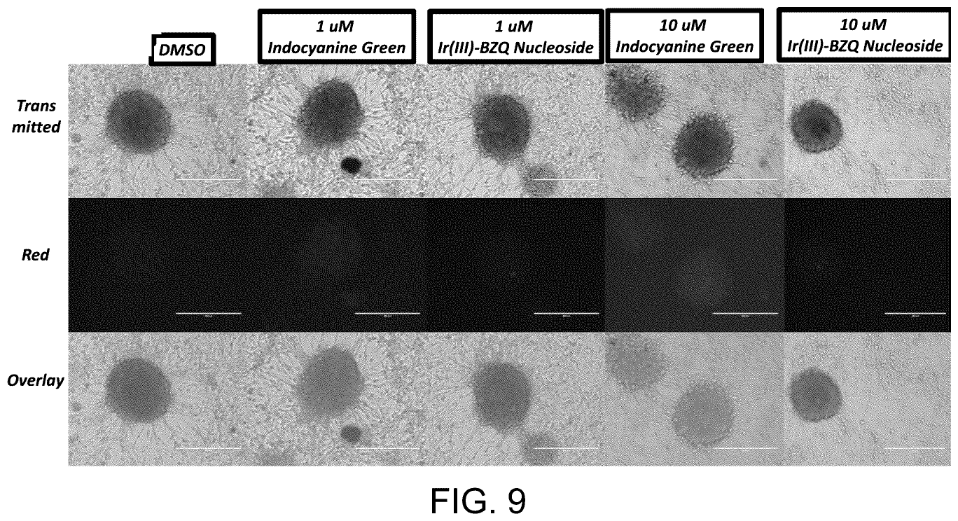

FIG. 9 shows a dose-and time dependent increase in red fluorescence in U87 cells (glioblastoma multiforme) treated with 1 and 10 μM Indocyanine green after 24 hours. Magnification is 10×. Direct comparison with cells treated with identical concentrations of Ir(III) BZQ nucleoside reveals that Indocyanine green possesses ˜5 times higher fluorescence emission activity in the red spectral range (600-700 nm).

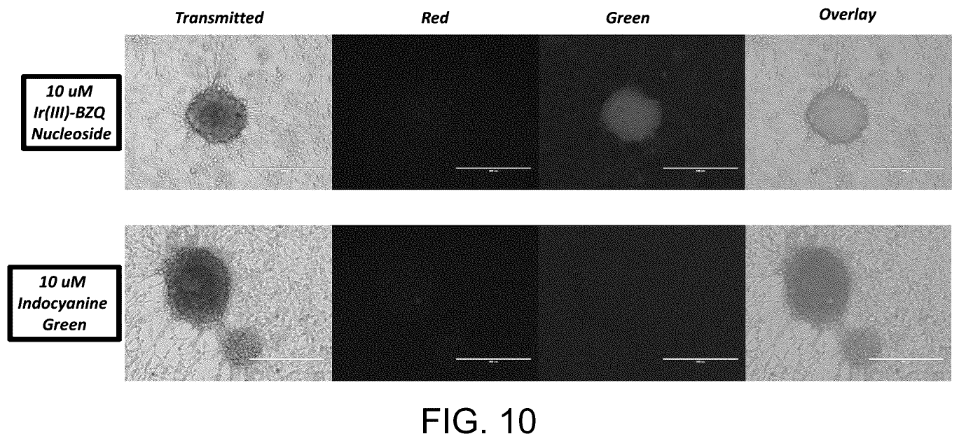

FIG. 10 compares fluorescence activity of Ir(III)-BZQ nucleoside with Indocyanine green at a fixed concentration of 10 μM. Images were taken comparing fluorescence emission in the red (600-700 nm) and green spectral range (500-600 nm) after 24 hours post-treatment. Direct comparison of the images show that Indocyanine green possesses superior fluorescence emission activity in the red spectral range (600-700 nm). However, Ir(III) BZQ nucleoside displays superior fluorescence emission in the green spectral range (500-600 nm) as compared to Indocyanine green. Thus, Ir(III) BZQ nucleoside possesses distinct spectral characteristics that confirm its utility as a tumor margin detection agent, albeit at different ranges compared to Indocyanine green.

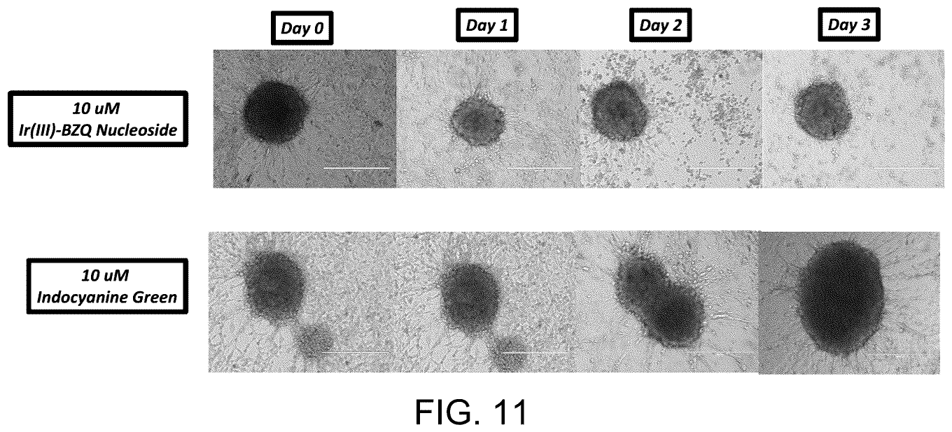

Imaging data provided in FIG. 11 indicates that Ir(III) BZQ nucleoside possesses cell-killing activity whereas Indocyanine green does not. The overall size of the 3D masses of U87cells treated with 10 μM Indocyanine green increases as a function of time (Δt=1 to 3 days treatment). In addition, the density of 2D cells increases as a function of time. Collectively, these data indicate that Indocyanine green functions solely as a diagnostic agent and possesses no detectable therapeutic activity. In contrast, imaging data provided in FIG. 11 shows that Ir(III)-BZQ nucleoside possesses cell-killing activity. Thus, Ir(III)-BZQ nucleoside possesses both diagnostic activity and therapeutic cell-killing activity, making it a unique theranostic agent with clinical utility in oncology.

### Theranostic Activity of Ir(III)-BZQ Nucleoside Against Triple Negative Breast Cancer

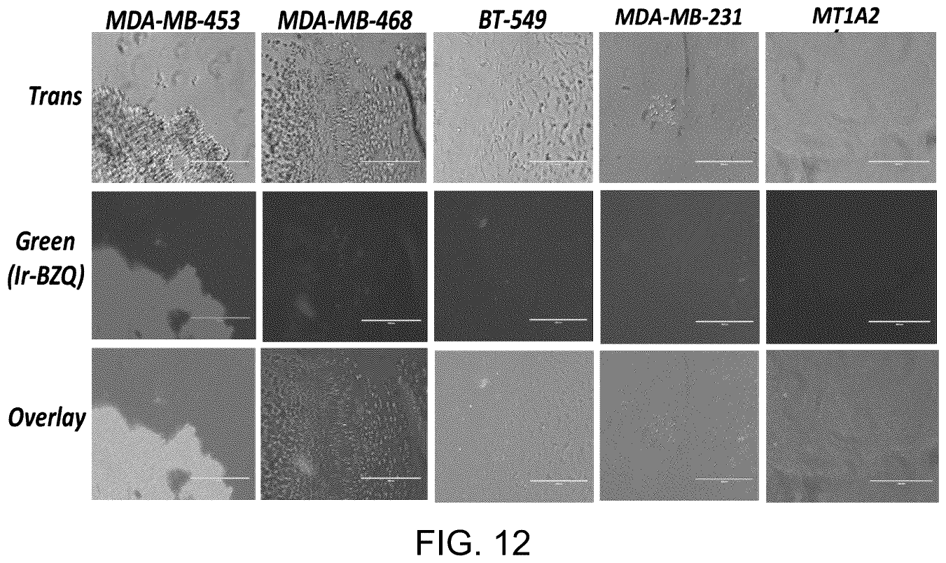

The theranostic activity of Ir(II)-BZQ nucleoside was also confirmed using 3D models of triple negative breast cancer (TNBC) (MDA-MB-453, MDA-MB-468, BT-549, MDA-MB-231). TNBC cells were chosen due to the poor prognosis caused by their aggressive nature and limited treatment options. 3D cancer cells were treated with 10 μM Ir(III)-BZQ nucleoside for 24 hours prior to microscopy analyses. FIG. 12 shows that MB-453 and MB-468 cells display high fluorescence signals indicating efficient uptake and accumulation of Ir(III)-BZQ nucleoside. In contrast, BT-549 cells and MB-231 show weak fluorescence while MT1A2 cells (derived from PyMT) show no fluorescence signals.

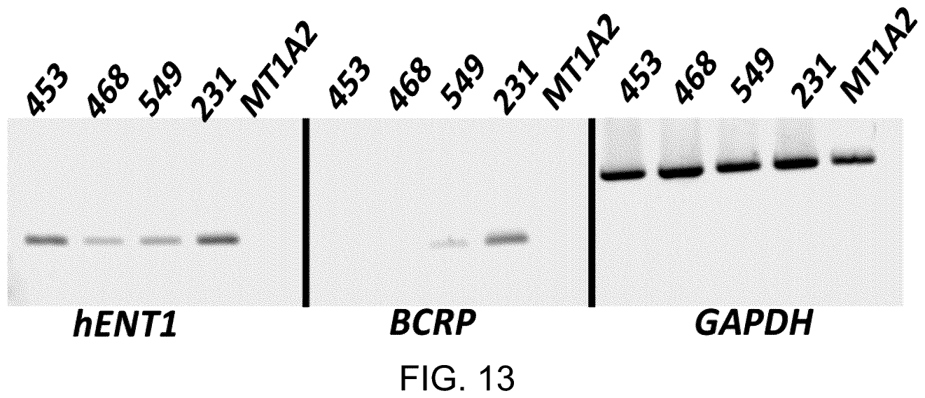

Differences in Ir(III)-BZQ nucleoside accumulation was interrogated by examining expression levels of two key gene products, human equilibrative nucleoside transporter 1 (hENT1) needed for uptake and breast cancer resistance protein (BCRP) that may facilitate efflux. Data provided in FIG. 13 show that MDA-MB-453 and MDA-MB-468 cells (high accumulation) express hENT1 but not BCRP. This result suggests that hENT1 activity is necessary for efficient uptake of Ir(III)-BZQ nucleoside. In contrast, BT-549 and MDA-MB-231 cells (low accumulation) show modest mRNA expression for hENT1 but higher levels of BCRP. This suggests that low accumulation reflects the ability of BCRP to export Ir(III)-BZQ nucleoside after it is imported by hENT1. Finally, MT1A2 cells (no accumulation) do not express mRNA for either hENT1 or BCRP. The absence of hENT1 explains the lack of Ir(III) BZQ nucleoside uptake. Collectively, these results provide reasonable mechanisms accounting for observed differences in Ir(III)-BZQ nucleoside accumulation. Both proteins could function as predictive biomarkers to stratify patients as “responders” (hENT1 positive/BCRP negative) or “non-responders” (hENT1 negative/BCRP positive).

The therapeutic activity and selectivity of Ir(III)-BZQ nucleoside was confirmed by measuring LD₅₀ values against MB-468 cells (high accumulation), MDA-MB-231 (low accumulation), and normal fibroblasts using 2D cell cultures (Table 1, below). Ir(III)-BZQ-nucleoside displays cytotoxic effects in the low micromolar range against MDA-MB-468 cells (high accumulation) while producing minimal cytotoxic effects against MDA-MB-231 (low accumulation) and normal fibroblasts. These data provide a reasonable correlation between Ir(III)-BZQ nucleoside uptake and accumulation with its potency and selectivity as an anti-cancer agent. Finally, reduced potency of Ir(III)-BZQ nucleoside against non-cancerous fibroblasts suggests selectivity for killing cancerous cells compared to healthy, normal cells.

Dual parameter flow cytometry employing Annexin V and propidium iodide staining was used to measure apoptosis in MDA-MB-468 cells treated with variable concentrations of Ir(III)-BZQ nucleoside. Results demonstrate an increase in both early- and late-stage apoptosis without causing necrotic cell death. Microscopy imaging verifies an increase in green fluorescence caused by the accumulation of Ir(III)-BZQ nucleoside and correlates with the dose-dependent increase in apoptosis caused by the agent.

### Imaging Activity of Two (2) Additional Ir(III)-Nucleosides Against Triple Negative Breast Cancer

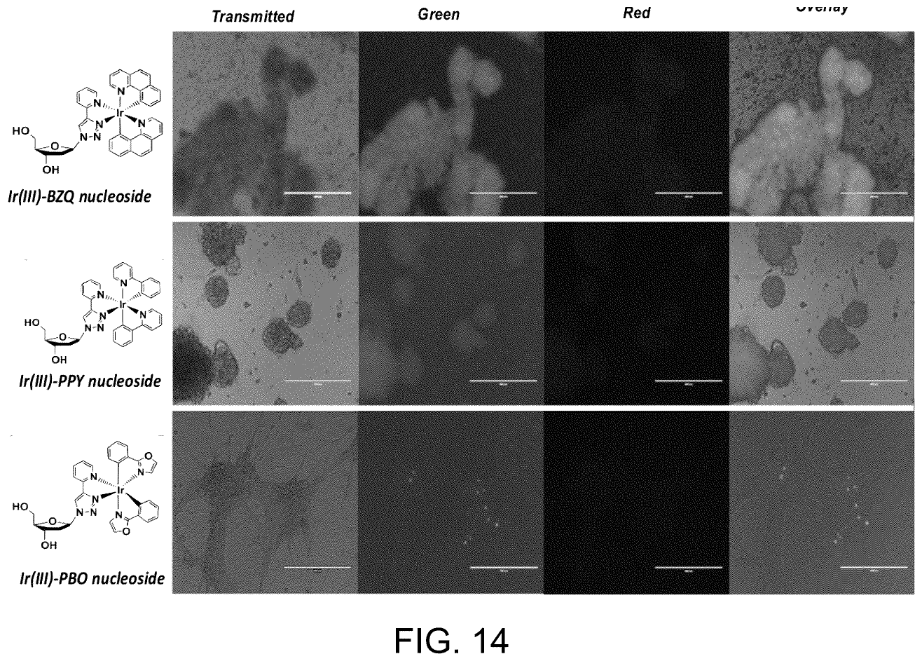

Two (2) new Iridium-nucleosides designated Ir(III)-PPY-nucleoside (small, hydrophobic) and Ir(III)-PBO nucleoside (small, hydrophilic) were synthesized and tested as cancer imaging agents. FIG. 14 compares the imaging properties of these two analogs versus our lead agent, Ir(III)-BZQ nucleoside (small, hydrophobic). Experiments were performed treating MDA-MB-453 cells with 1 μM of each compound for 3 days. Imaging data show that Ir(III)-BZQ nucleoside displays superior activity among the three compounds as it has a ˜5-fold higher fluorescence intensity compared to Ir(III)-PPY nucleoside and Ir(III)-PBO nucleoside. In addition, cells treated with Ir(III)-PBO nucleoside show unique punctates. This phenomenon precludes its further development as a tumor margin imaging agent as it poorly defines the entire 3D mass as compared to Ir(III)-BZQ nucleoside and Ir(III)-PPY nucleoside.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. A process of treating a cancer patient, the process comprising:
administering an iridium-containing nucleoside to the patient;
obtaining a medical image of the patient; and
excising a tumor and a tumor margin from the patient, wherein an amount of tissue excised is based on identifying cellular uptake of the iridium-containing nucleoside from the medical image.

2. The process of claim 1, wherein the administration of the iridium-containing nucleoside comprises intravenous administration.

3. The process of claim 1, wherein the administration of the iridium-containing nucleoside comprises local administration

4. The process of claim 1, wherein the medical image is obtained by providing real-time information simultaneously by combining color reflectance images with fluorescence emission from the iridium-containing nucleoside.

5. The process of claim 1, wherein the iridium-containing nucleoside is selected from the group consisting of:

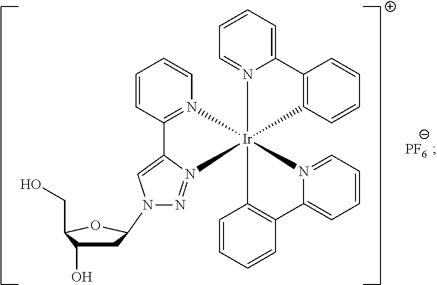

6. The process of claim 1, wherein the iridium-containing nucleoside is:

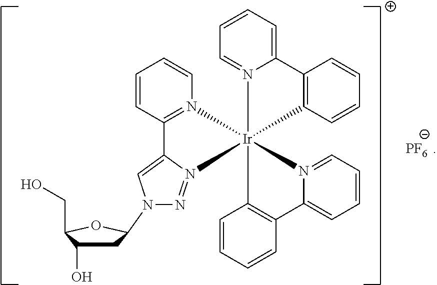

7. The process of claim 1, wherein the iridium-containing nucleoside is:

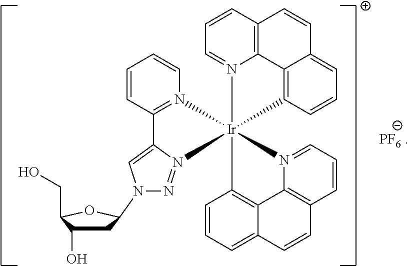

8. The process of claim 1, wherein the iridium-containing nucleoside is:

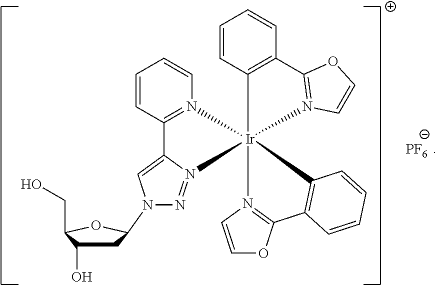

9. The process of claim 1, wherein the obtaining and excising are performed simultaneously during a surgery.

10. The process of claim 1, wherein the obtaining is performed from about 4 to about 24 hours after administering the iridium-containing nucleoside.

11. The process of claim 1, wherein the iridium-containing nucleoside is a structural analog of deoxyadenosine.

12. The process of claim 1, wherein the iridium-containing nucleoside comprises a pyridyl triazole linking iridium to deoxyribose.

13. The process of claim 1, wherein the tumor is a brain tumor, a breast tumor, or an ovarian tumor.

14. The process of claim 1, wherein the tumor is a brain tumor.

15. The process of claim 1, wherein the tumor is a breast tumor.

16. The process of claim 1, wherein the tumor is an ovarian tumor.

17. The process of claim 1, wherein the tumor is a brain tumor, a breast tumor, or an ovarian tumor; wherein the medical image is obtained by providing real-time information simultaneously by combining color reflectance images with fluorescence emission from the iridium-containing nucleoside; and wherein the iridium-containing nucleoside is selected from the group consisting of:

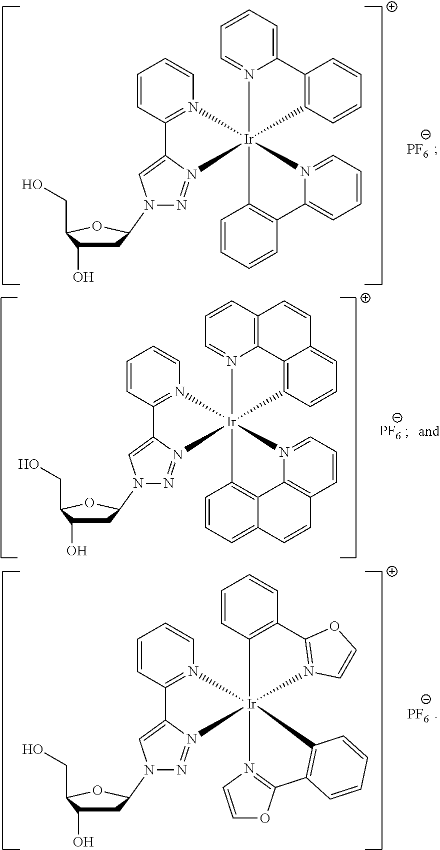

18. An iridium-containing nucleoside is selected from the group consisting of:

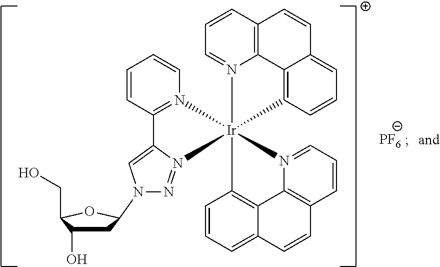

19. The iridium-containing nucleoside of claim 18, wherein the nucleoside is:

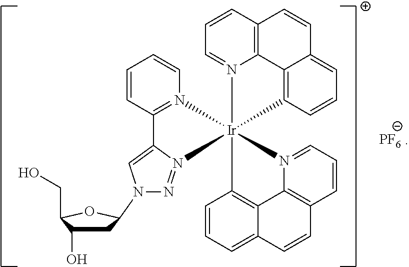

20. The iridium-containing nucleoside of claim 18, wherein the nucleoside is: [chemical structure]
